SEQUENCE LISTING (17 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 5 aa, Mus musculus
SYAMS

SEQ ID NO: 3 - protein, 16 aa, Mus musculus
SISKGGNTYYPNSVKG

SEQ ID NO: 4 - protein, 10 aa, Mus musculus
GWGDYGWFAY

SEQ ID NO: 5 - protein, 11 aa, Mus musculus
KASQDINRYLN

SEQ ID NO: 6 - protein, 7 aa, Mus musculus
RANRLLD

SEQ ID NO: 7 - protein, 9 aa, Mus musculus
LQYDEFPLT

SEQ ID NO: 8 - protein, 118 aa, Mus musculus
EVKLVESGGDLVKPGGSLKLSCAASGFTFSSYAMSWVRQNPEKRLEWVASISKGGNTYYPNSVKGRFTISRDNARNILYLQMSSLRSEDTALYYCARGWGDYGWFAYWGQVTLVTVSA

SEQ ID NO: 9 - protein, 107 aa, Mus musculus
DIKMTQSPSSMYASLGERVTITCKASQDINRYLNWFQQKPGKSPKTLIYRANRLLDGVPSRFSGSGSGQDYSLTISSLDYEDMGIYYCLQYDEFPLTFGDGTKLELK

SEQ ID NO: 10 - protein, 10 aa, Mus musculus
GFTFRSYGMS

SEQ ID NO: 11 - protein, 17 aa, Mus musculus
TINSDGSYTYYPDSVKG

SEQ ID NO: 12 - protein, 8 aa, Mus musculus
SWDGAMDY

SEQ ID NO: 13 - protein, 16 aa, Mus musculus
RSSQSILHSNGNTYFE

SEQ ID NO: 14 - protein, 7 aa, Mus musculus
KVSNRFS

SEQ ID NO: 15 - protein, 9 aa, Mus musculus
FQGSLVPWT

SEQ ID NO: 16 - protein, 117 aa, Mus musculus
EVKLVESGGDLVKPGGSLKLSCAASGFTFRSYGMSWVRQTPDKRLEWVTTINSDGSYTYYPDSVKGRFTISRDNAKNTLYLQMSSLKSEDTAMYYCAVSWDGAMDYWGQGTSVTVSS

SEQ ID NO: 17 - protein, 112 aa, Mus musculus
DVLVTQTPLSLPVSLGDQASISCRSSQSILHSNGNTYFEWYLQRPGQSPKLLIYKVSNRFSGVPDRFSGSGSGTDFTLKISRVEAEDLGVYYCFQGSLVPWTFGGGTKLEIK

(19) United States
(12) Patent Application Publication
(10) Pub. No.: US 2025/0362311 A1
(43) Pub. Date: Nov. 27, 2025

(54) BLOOD-BASED SCREENING OF SUBJECTS FOR A CLINICAL TRIAL FOR TREATMENT OF TAUOPATHY OR AMYLOIDOGENIC DISEASE

(71) Applicant: Janssen Pharmaceutica NV, Beerse (BE)

(72) Inventors: Gallen TRIANA-BALTZER, San Diego, CA (US); Ziad Serhal Saad, San, CA (US)

(21) Appl. No.: 18/875,041

(22) PCT Filed: Jun. 15, 2023
(86) PCT No.: PCT/EP2023/066105

§ 371 Date: Dec. 13, 2024

Related U.S. Application Data
(60) Provisional application No. 63/366,449, filed on Jun. 15, 2022.
(60) Provisional application No. 63/437,260, filed on Jan. 5, 2023.

(51) Int. Cl. G01N 33/68

(52) U.S. Cl. CPC G01N 33/6896; G01N 2800/2814

(57) ABSTRACT

A method of pre-screening a human subject for a clinical trial for treatment of tauopathy or an amyloidogenic disease. The method comprises obtaining a plasma sample from the subject and determining a concentration of p217+tau present in the plasma sample. The method further comprises indicating the subject for further screening for the clinical trial when the concentration of p217+tau present in the plasma sample is greater than or equal to a minimum threshold and less than or equal to a maximum threshold. The minimum threshold corresponds to an amount of p217+tau in plasma over which subjects present with mild cognitive impairment (MCI) and an increased accumulation of tau tangles in the brain as compared to a cognitively normal patient. The maximum threshold corresponds to an amount of p217+tau present in plasma over which subjects present with pathology of widespread accumulation of tau tangles in multiple regions of the brain.

### CROSS-REFERENCE TO RELATED APPLICATIONS

This application claims the benefit of U.S. Provisional Application No. 63/366,449, filed on Jun. 15, 2022, and U.S. Provisional Application No. 63/437,260, filed on Jan. 5, 2023, each of which is incorporated herein by reference in its entirety.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JAB7165WOPCTISEQLIST.xml”, creation date of Jun. 5, 2023, and a size of 17 kilobytes (KB). This sequence listing submitted is part of the specification and is herein incorporated by reference in its entirety.

### FIELD OF INVENTION

The present application relates to methods for blood-based screening of subjects for a clinical trial for treatment of tauopathy or amyloidogenic disease. In particular, the present application relates to methods of identifying patients suitable for a clinical trial for treatment of tauopathy or amyloidogenic disease based on measuring an amount of singly-or multiply-phosphorylated p217+tau protein species in a blood-based sample and uses thereof.

### BACKGROUND OF INVENTION

Alzheimer's Disease (AD) is a degenerative brain disorder characterized clinically by progressive loss of memory, cognition, reasoning, judgment, and emotional stability that gradually leads to profound mental deterioration and ultimately death. AD is a very common cause of progressive mental failure (dementia) in aged humans. More than 5 million people in the United States are living with AD, and the number is growing with an aging population. Indeed, 10% of people over age 65 have AD, and it is the 5th leading cause of death in this population. Overall AD is the 6th leading cause of death in the United States (1 in 3 seniors die with AD or another dementia), and it is estimated to cost the US $305 billion in 2020. AD has also been observed in ethnic groups worldwide and presents a major present and future public health problem.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles of paired helical filaments, are generally found in several areas of the human brain important for memory and cognitive function in patients with AD.

The progression of tauopathy in an AD brain follows distinct spreading patterns. A tauopathy transmission and spreading hypothesis has been described based on the Braak stages of tauopathy progression in the human brain and tauopathy spreading after tau aggregate injections in preclinical tau models (Frost et al., J Biol Chem. 284:12845-52, 2009; Clavaguera et al., Nat Cell Biol. 11:909-13, 2009). It is believed that tauopathy can spread in a prion-like fashion from one brain region to the next. This spreading process would involve an externalization of tau seeds that can be taken up by nearby neurons and induce further tauopathy.

There is currently a significant unmet need for clinically effective, safe treatments for tauopathy or amyloidogenic disease, such as, for example, Alzheimer's disease. However, any investigational medicinal product for treatment of tauopathy or amyloidogenic disease requires clinical trial studies in suitable subjects to demonstrate efficacy in ameliorating or slowing progression of tauopathy or amyloidogenic disease.

### BRIEF SUMMARY OF THE INVENTION

One exemplary embodiment of the present application is directed to a method of pre-screening a human subject for a clinical trial for treatment of tauopathy or an amyloidogenic disease. The method comprises obtaining a plasma sample from the subject and determining a concentration of p217+tau present in the plasma sample. For example, the concentration of p217+tau present in the plasma sample may be determined by contacting the plasma sample with a capture antibody directed against a p217+tau epitope to bind the capture antibody to p217+tau peptides in the plasma sample to form antibody-peptide complexes, contacting the antibody-peptide complexes with a detection antibody to bind the detection antibody to the antibody-peptide complexes, and detecting the detection antibody to determine an amount of the p217+tau peptides in the plasma sample. In one example, the concentration of p217+tau is determined using an assay having a LLOQ<0.04 pg/ml.

The method further comprises indicating the subject for further screening for the clinical trial when the concentration of p217+tau present in the plasma sample is greater than or equal to a minimum threshold and less than or equal to a maximum threshold. The minimum threshold corresponds to an amount of p217+tau in plasma over which subjects present with mild cognitive impairment (MCI) and an increased accumulation of tau tangles in the brain as compared to a cognitively normal patient. In one example, the minimum threshold is from about 0.075 pg/ml to about 0.125 pg/ml. The maximum threshold corresponds to an amount of p217+tau present in plasma over which subjects present with pathology of widespread accumulation of tau tangles in multiple regions of the brain. In one example, the maximum threshold is from about 0.225 pg/ml to about 0.275 pg/ml. More specifically, the indicating step comprises indicating the subject for further screening for the clinical trial when the concentration of p217+tau present in the plasma sample is ≥0.1 pg/ml and ≤0.25 pg/ml.

In another aspect of the present application, a method of screening a human subject for a clinical trial for treatment of tauopathy or an amyloidogenic disease is provided. The method comprises obtaining a plasma sample from the subject and determining a concentration of p217+tau present in the plasma sample. The method also comprises obtaining PET data of the brain of the subject generated with a tau-specific or amyloid-specific radioactive tracer when the concentration of p217+tau present in the plasma sample is greater than or equal to a minimum threshold and less than or equal to a maximum threshold. The minimum threshold corresponds to an amount of p217+tau in plasma over which subjects present with mild cognitive impairment (MCI) and an increased accumulation of tau tangles in the brain as compared to a cognitively normal patient. In one example, the minimum threshold is from about 0.075 pg/ml to about 0.125 pg/ml. The maximum threshold corresponds to an amount of p217+tau present in plasma over which subjects present with pathology of widespread accumulation of tau tangles in multiple regions of the brain. In one example, the maximum threshold is from about 0.225 pg/ml to about 0.275 pg/ml. More specifically, the indicating step comprises indicating the subject for further screening for the clinical trial when the concentration of p217+tau present in the plasma sample is ≥0.1 pg/ml and ≤0.25 pg/ml. The method further comprises analyzing the PET data to determine whether the subject is tau positive or amyloid positive, and whether the subject has widespread tau tangles or widespread amyloid fibrils. The method further comprises indicating the subject as suitable for inclusion in the clinical trial when: (i) the concentration of p217+tau present in the plasma sample is greater than or equal to the minimum threshold and less than or equal to the maximum threshold, (ii) the PET data indicates the subject is tau positive or amyloid positive, and (iii) the PET data indicates the subject does not have without having widespread tau tangles or widespread amyloid fibrils.

These and other aspects of the invention will become apparent to those skilled in the art after a reading of the following detailed description of the invention, including the figures and appended claims.

### BRIEF DESCRIPTION OF THE DRAWINGS

FIG. 1 shows an exemplary method for pre-screening or screening a human subject for a clinical trial for treatment of tauopathy or an amyloidogenic disease.

FIG. 2 shows another exemplary method for pre-screening or screening a human subject for a clinical trial for treatment of tauopathy or an amyloidogenic disease.

FIG. 3a shows data for precision (% CV) of an exemplary p217+tau assay across different concentrations of p217+tau detected from plasma samples collected from 832 patients screened for a clinical trial.

FIG. 3b shows a distribution of concentrations of p217+tau detected by an exemplary p217+tau assay from plasma samples collected from 831 patients screened for a clinical trial.

FIG. 4 shows Braak ROIs.

FIG. 5 shows significant (p<10⁻¹⁵) correlations between plasma p217+tau and MK6240 across all six (6) Braak ROIs.

FIG. 6 shows that plasma p217+tau can predict in-vivo Braak stage of 3 or lower with AUC of 82%.

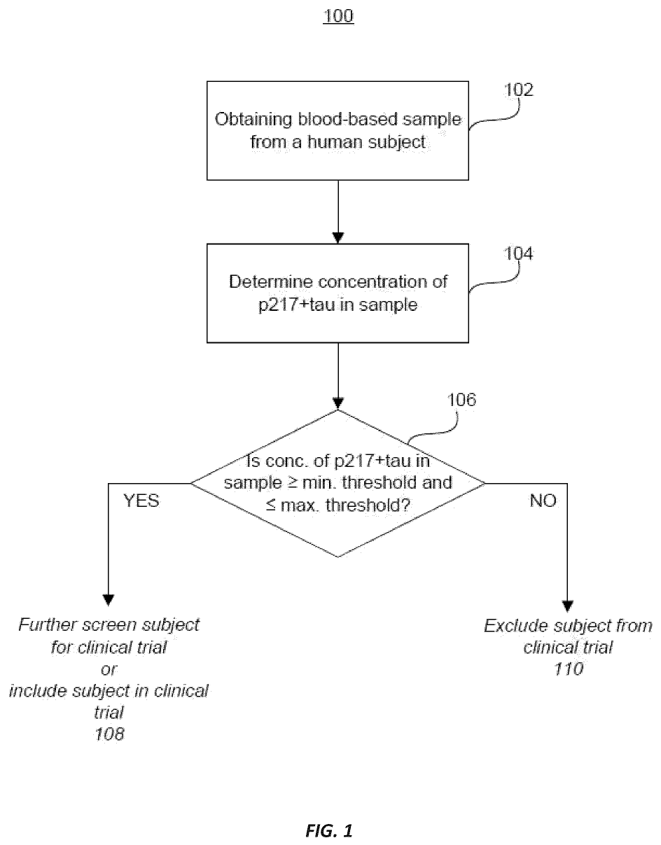

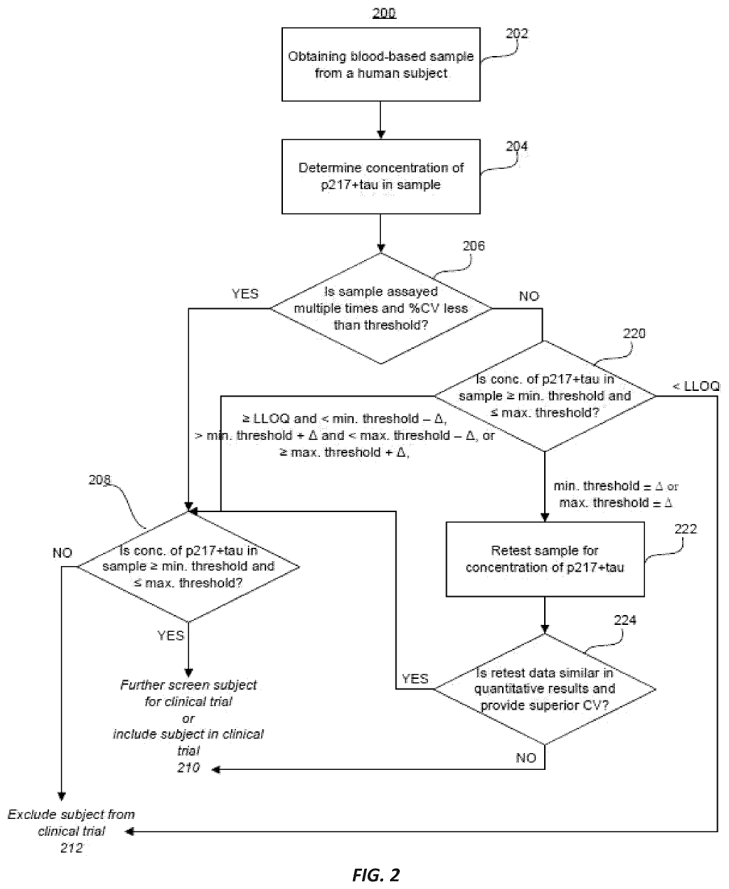

### DETAILED DESCRIPTION OF THE INVENTION

Unless defined otherwise, all technical and scientific terms used herein have the same meaning commonly understood to one of ordinary skill in the art to which this invention pertains. Otherwise, certain terms used herein have the meanings as set in the specification. All patents, published patent applications and publications cited herein are incorporated by reference as if set forth fully herein. It is noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the term “antibody” or “immunoglobulin” refers to a specific protein capable of binding an antigen or portion thereof. These terms are used herein in a broad sense and includes immunoglobulin or antibody molecules including polyclonal antibodies, monoclonal antibodies (including murine, human, human-adapted, humanized and chimeric monoclonal antibodies) and antibody fragments.

In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Accordingly, the antibodies of the present application can be of any of the five major classes or corresponding sub-classes. Preferably, the antibodies of the present application are IgG1, IgG2, IgG3 or IgG4. Antibody light chains of any vertebrate species can be assigned to one of two clearly distinct types, namely kappa and lambda, based on the amino acid sequences of their constant domains. Accordingly, the antibodies of the present application can contain a kappa or lambda light chain constant domain. According to particular embodiments, the antibodies of the present application include heavy and/or light chain constant regions from mouse antibodies or human antibodies.

In addition to the heavy and light constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat: “Complementarity Determining Regions” or “CDRs” are based on sequence variability (Wu and Kabat, J Exp Med. 132:211-50, 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia: The term “hypervariable region,” “HVR” refers to the regions of an antibody variable domain which are hypervariable in structure as defined by Chothia and Lesk (Chothia and Lesk, J Mol Biol. 196:901-17, 1987). Generally, the antigen-binding site has three hypervariable regions in each VH (H1, H2, H3) and VL (L1, L2, L3). Numbering systems as well as annotation of CDRs and HVRs have been revised by Abhinandan and Martin (Abhinandan and Martin, Mol Immunol. 45:3832-9, 2008);
(iii) IMGT: Another definition of the regions that form the antigen-binding site has been proposed by Lefranc (Lefranc et al., Dev Comp Immunol. 27:55-77, 2003) based on the comparison of V domains from immunoglobulins and T-cell receptors. The International ImMunoGeneTics (IMGT) database (http://www_imgt_org) provides a standardized numbering and definition of these regions. The correspondence between CDRs, HVRs and IMGT delineations is described in Lefranc et al., 2003, Id.;
(iv) The antigen-binding site can also be delineated based on “Specificity Determining Residue Usage” (SDRU) (Almagro, Mol Recognit. 17:132-43, 2004), where SDR, refers to amino acid residues of an immunoglobulin that are directly involved in antigen contact.

“Framework” or “framework sequence” is the remaining sequences within the variable region of an antibody other than those defined to be antigen-binding site sequences. Because the exact definition of an antigen-binding site can be determined by various delineations as described above, the exact framework sequence depends on the definition of the antigen-binding site. The framework regions (FRs) are the more highly conserved portions of variable domains. The variable domains of native heavy and light chains each comprise four FRs (FR1, FR2, FR3 and FR4, respectively) which generally adopt a beta-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227:799-817, 1992; Tramontano et al., J. Mol. Biol. 215:175-182, 1990). Despite their high sequence variability, five of the six loops adopt just a small repertoire of main-chain conformations, called “canonical structures.” These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “epitope” refers to a site on an antigen to which an immunoglobulin, antibody, or antigen-binding fragment thereof, specifically binds. Epitopes can be formed both from contiguous amino acids or from noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

As used herein, the term “tau” or “tau protein” refers to an abundant central and peripheral nervous system protein having multiple isoforms. In the human central nervous system (CNS), six major tau isoforms ranging in size from 352 to 441 amino acids in length exist due to alternative splicing (Hanger et al., Trends Mol Med. 15:112-9, 2009). The isoforms differ from each other by the regulated inclusion of 0-2 N-terminal inserts, and 3 or 4 tandemly arranged microtubule-binding repeats, and are referred to as 0N3R, 1N3R, 2N3R, 0N4R, 1N4R and 2N4R. As used herein, the term “control tau” refers to the tau isoform of SEQ ID NO: 1 that is devoid of phosphorylation and other post-translational modifications. As used herein, the term “tau” includes proteins comprising mutations, e.g., point mutations, fragments, insertions, deletions and splice variants of full-length wild type tau. The term “tau” also encompasses post-translational modifications of the tau amino acid sequence. Post-translational modifications include, but are not limited to, phosphorylation.

Unless otherwise indicated, as used herein, the numbering of the amino acid in a tau protein or fragment thereof is with reference to the amino acid sequence set forth in SEQ ID NO: 1.

As used herein, the term “p217+tau peptides,” “p217+tau,” or “p217+tau protein” means a human tau protein or tau fragment that is phosphorylated at residue 217 (pT217) of tau protein, and may or may not be further phosphorylated at additional residues, such as, for example, residue 212 (pT212) of tau protein, wherein the numbering of the positions is according to the numbering in SEQ ID NO: 1.

As used herein, the term “p217+tau epitope” refers to a tau epitope containing at least one of phosphorylated T217 and phosphorylated T212, wherein the numbering of the positions is according to the numbering in SEQ ID NO: 1. Examples of p217+tau epitope include, e.g., a pT3 epitope. As used herein, the term “pT3 epitope” refers to an epitope containing amino acids 210-220 of human tau protein that is phosphorylated at residue 217, and may or may not be further phosphorylated at additional residues, such as, for example, residue 212, wherein the numbering of the positions is according to the numbering in SEQ ID NO: 1.

As used herein, the term “capture antibody” refers to an antibody that binds to an antigen of interest and is directly or indirectly linked to a solid support. Examples of solid supports include, but are not limited to, microparticles or beads, such as a magnetic beads or paramagnetic beads. Examples of capture antibodies include, but are not limited to, a monoclonal antibody that binds to a p217+tau epitope.

As used herein, the term “detection antibody” refers to an antibody that binds to an antigen of interest and has a detectable label or is linked to a secondary detection system. Examples of detectable labels include, but are not limited to, various enzymes, prosthetic groups, fluorescent materials, luminescent materials, bioluminescent materials, and radioactive materials. Examples of detection antibodies include, but are not limited to, a monoclonal antibody that binds to tau protein, preferably an epitope comprising amino acids 7-20 or 116-127 of human tau protein, wherein the numbering of the positions is according to the numbering in SEQ ID NO: 1. When a monoclonal antibody that binds to a tau protein at an epitope comprising amino acids 7-20 is used as a detection antibody for captured p217+tau peptides, long tau fragments are detected. When a monoclonal antibody that binds to a tau protein at an epitope comprising amino acids 116-127 is used as a detection antibody for captured p217+tau peptides, both short and long tau fragments are detected.

As used herein, the term “subject” refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig, marmoset or mouse) or a primate (e.g., a monkey, chimpanzee, or human). In particular embodiments, the subject is a human.

As used herein a “tauopathy” encompasses any neurodegenerative disease that involves the pathological aggregation of tau within the brain. In addition to familial and sporadic AD, other exemplary tauopathies are frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, and chronic traumatic encephalopathy, such as dementia pugulistica (boxing disease) (Morris et al., Neuron, 70:410-26, 2011).

As used herein, the term “amyloidogenic disease” includes any disease associated with (or caused by) the formation or deposition of insoluble amyloid fibrils. Exemplary amyloidogenic diseases include, but are not limited to systemic amyloidosis, Alzheimer's disease, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. Accordingly, Alzheimer's disease is an example of a “disease characterized by deposits of Aβ” or a “disease associated with deposits of Aβ”, e.g., in the brain of a subject or patient. The terms “β-amyloid protein,” “β-amyloid peptide,” “β-amyloid,” “Aβ” and “Aβ peptide” are used interchangeably herein.

As used herein, the terms “determining,” “measuring,” “assessing,” and “assaying” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present, as well as determining whether it is present or absent.

As used herein, the term “diagnosis” means detecting a disease or disorder or determining the stage or degree of a disease or disorder, such as a tauopathy or an amyloidogenic disease. Usually, a diagnosis of a disease or disorder is based on the evaluation of one or more factors and/or symptoms that are indicative of the disease. A diagnosis can be made based on the presence, absence or amount of a factor which is indicative of presence or absence of the disease or condition, e.g., p217+tau. Each factor or symptom that is considered to be indicative for the diagnosis of a particular disease does not need be exclusively related to the particular disease, i.e., there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, e.g., an anti-p217+tau antibody therapy, or predicting the pattern of response to a drug therapy, e.g., an anti-p217+tau antibody therapy. The diagnostic methods may be used independently, or in combination with other diagnosing and/or staging methods known in the medical arts for a particular disease or disorder, e.g., Alzheimer's disease.

As used herein, the terms “increase” and “decrease” refer to differences in the quantity of a particular biomarker in a sample as compared to a control or reference level. For example, the quantity of particular peptide, may be present at an elevated amount or at a decreased amount in samples of patients with a disease compared to a reference level. In one embodiment, an “increase of a level” or “decrease of a level” may be a difference between the level of biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, an “increase of a level” or “decrease of a level” may be a statistically significant difference between the level of the biomarker present in a sample as compared to a control. For example, a difference may be statistically significant if the measured level of the biomarker falls outside of about 1.0 standard deviation, about 1.5 standard deviations, about 2.0 standard deviations, or about 2.5 standard deviations of the mean of any control or reference group. The reference or control can be, for example, a sample from a healthy individual, or a sample taken from the same individual at an earlier time point, such as a time point prior to administration of a therapeutic or an earlier time point during a therapeutic regimen.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

An “isolated antibody that binds to a tau protein” or an “isolated anti-tau antibody”, as used herein, is intended to refer to an antibody that specifically binds tau protein and which is substantially free of other antibodies having different antigenic specificities (for instance, an isolated anti-tau detection antibody is substantially free of antibodies that specifically bind antigens other than tau). An isolated anti-tau detection antibody can, however, have cross-reactivity to other related antigens, for instance from other species (such as tau species homologs).

As used herein, the term “specifically binds” or “specific binding” refers to the ability of an anti-tau antibody of the present application to bind to a predetermined target with a dissociation constant (K_{D}) of about 1×10⁻⁶ M or tighter, for example, about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. The K_{D} is obtained from the ratio of Kd to Ka (i.e., Kd/Ka) and is expressed as a molar concentration (M). K_{D} values for antibodies can be determined using methods in the art in view of the present disclosure. For example, the K_{D} value of an anti-tau antibody can be determined by using surface plasmon resonance, such as by using a biosensor system, e.g., a Biacore® system, a Proteon instrument (BioRad), a KinExA instrument (Sapidyne), ELISA or competitive binding assays known to those skilled in the art. Typically, an anti-tau antibody binds to a predetermined target (i.e., tau) with a K_{D} that is at least ten fold less than its K_{D} for a nonspecific target as measured by surface plasmon resonance using, for example, a Proteon Instrument (BioRad). The anti-tau antibodies that specifically bind to tau can, however, have cross-reactivity to other related targets, for example, to the same predetermined target from other species (homologs), such as from mouse, rat, marmoset, dog or pig.

As used herein, the term “accuracy” refers to the degree of closeness of a valued to the true value of an assay.

As used herein, the term “precision” refers to closeness of agreement among a series of measurements obtained from multiple samplings of the same homogenous sample of an assay.

The term “sensitivity” as used herein refers to the lowest analyte concentration in a sample that can be measured with acceptable accuracy and precision in an assay.

In conducting a clinical trial of a treatment for tauopathy or an amyloidogenic disease, there is a need to screen human subjects who are seeking to enroll in the clinical trial and select those subjects who are in need of such treatment, such as, those who have developed and/or are at risk of developing tauopathy or an amyloidogenic disease, for inclusion in the clinical trial. Therefore, the subject needs to meet a minimum level of disease progression of tauopathy and/or amyloidogenic disease to be included in the clinical trial. Additionally, it is believed that anti-tau therapies and anti-amyloid are most effective in treating tauopathy and amyloidogenic disease, respectively, when treatment is initiated early in the progression of disease. Therefore, it may also be desirable to select those subjects who are not at late disease progression of tauopathy or amyloidogenic disease so that the treatment is more likely to have an effect and/or any effect by the treatment for tauopathy or an amyloidogenic disease under clinical trial for slowing progression of tauopathy or an amyloidogenic disease can be readily observed. To identify those subjects who are in need of a treatment for tauopathy or an amyloidogenic disease and not at late disease progression of tauopathy and/or amyloidogenic disease, it may be desirable to select those subjects who are above a minimum level of disease progression of tauopathy and/or amyloidogenic disease and below a maximum level of disease progression of tauopathy and/or amyloidogenic disease before progressing to further screening tests or for inclusion in the clinical trial.

The present application provides methods for pre-screening or screening a human subject for a clinical trial of a treatment for tauopathy or an amyloidogenic disease using an assay for detecting singly-or multiply-phosphorylated p217+tau peptides in blood-based samples. Accurate, sensitive, and precise measurement of pTau isoforms in plasma is believed to be a promising non-invasive method for detecting aberrant amyloid and tau processes. Longer fragments of multiphosphorylated tau, containing pT217 is believed to be one of the isoforms most associated with tauopathy and amyloidogenic disease, specifically, Alzheimer's disease pathology and may begin accumulating in CSF and plasma 10-20 years before cognitive decline. Phosphorylated tau, in particular, p217+tau, as measured in CSF and plasma is believed to be one of the most sensitive and specific biomarkers for tauopathy and amyloidogenic disease, specifically, Alzheimer's disease pathology and is believed to predict a “tau positive” pathology or a “amyloid positive” pathology (both are defined further below), as well as decline in cognitive state. It is believed that the amount of singly-or multiply-phosphorylated p217+tau peptides measured from a blood-based sample from a subject can serve as an indicator for progression of tauopathy and/or amyloidogenic disease in the subject. A lower concentration of p217+tau peptides measured from the sample corresponds to early disease progression of tauopathy and/or amyloidogenic disease, whereas a higher concentration of p217+tau peptides measured from the sample corresponds to late disease progression of tauopathy and/or amyloidogenic disease. Therefore, the amount of singly-or multiply-phosphorylated p217+tau peptides measured from a blood-based sample from subject may be useful as a pre-screening or screening criteria for determining whether the subject is suitable for inclusion in a clinical trial of a treatment for tauopathy or an amyloidogenic disease.

The pre-screening or screening methods described in the present application is useful for identifying suitable subjects for a clinical trial of a treatment for tauopathy or an amyloidogenic disease. The treatment for tauopathy may be any type of procedure or pharmaceutically active agent for treating or slowing progression of tauopathy or amyloidogenic disease. For example, the treatment for tauopathy under clinical trial may be a pharmaceutical composition comprising an active agent having potential to treat or slow progression of tauopathy, such as, anti-tau antibodies, anti-p217+tau antibodies, small interfering RNA (siRNA) against human tau, siRNA against p217+tau, etc. In another example, the treatment for amyloidogenic disease under clinical trial may be a pharmaceutical composition comprising an active agent having potential to treat or slow progression of the amyloidogenic disease, such as, anti-amyloid antibodies, beta secretase inhibitors, gamma secretase inhibitors, small interfering RNA (siRNA) against human β-amyloid, etc. In a particular example, the treatment under clinical trial is an active agent having potential to treat or slow progression of Alzheimer's disease.

Collection of blood-based samples, such as plasma, is fast and easy to perform and does not impose a significant discomfort or burden on to the subject. In contrast, other currently available tools for determining of a subject's progression of tauopathy and/or amyloidogenic disease, such as Alzheimer's disease, are costly and burdensome. For example, positron emission tomography using tau-specific radiotracers (Tau PET) has been used for measuring and locating tau neurofibrillary tangles pathology in the brain of patients. Similarly, positron emission tomography using amyloid-specific radiotracers (amyloid PET), for example, Aβ40 or Aβ42 specific radiotracers, has also been used for measuring and locating amyloid fibrils pathology in the brain of patients. However, both Tau PET and amyloid PET are costly and cumbersome procedures with limited availability of suitable tracers. Additionally, the process for imaging can be uncomfortable and therefore, more burdensome than collecting a blood sample for patients. Another exemplary tool for determining a subject's progression of tauopathy and/or amyloidogenic disease is the amount of phosphorylated tau peptides (e.g., singly- or multiply-phosphorylated p217+tau peptides) measured by an assay in a sample CSF retrieved from the subject. However, retrieval of CSF requires patients to undergo invasive lumbar puncture procedures involving physicians inserting a needle into the spinal canal to collect samples of CSF for use in assays. Such an invasive procedure can be painful and exposes the subject to risk of infection in the spinal canal. Therefore, the use of a blood-based assay, more specifically, a plasma-based assay, for detecting singly- or multiply-phosphorylated p217+tau peptides in a sample to pre-screen or screen human subjects for a clinical trial for the treatment of tauopathy or an amyloidogenic disease, in particular, Alzheimer's disease, provides an improved method to quickly and effectively identify those candidates who are most likely to fall within the desired clinical criteria (e.g., having a minimum level of disease progression of tauopathy and/or amyloidogenic disease but below a maximum level of disease progression of tauopathy and/or amyloidogenic disease) for enrolling into the clinical trial.

In one aspect of the present application, a method 100 for pre-screening or screening a human subject for a clinical trial for treatment of tauopathy or an amyloidogenic disease is provided. The method 100 comprises a first step 102 of obtaining a blood-based sample from the from a human subject. The blood-based sample may be a blood, serum, or plasma sample. Preferably, the sample is a plasma sample. More preferably, the plasma sample has not been immunoprecipitated to concentrate the p217+tau peptides contained therein. In a particular embodiment, the sample is a crude plasma sample.

In step 104, the sample may be further processed by any suitable means to concentrate the p217+tau peptides contained therein before analyzing the concentrated sample with an assay for determining an amount or concentration of p217+tau present in the sample. Preferably, in step 104, the sample is analyzed directly with an assay for determining an amount or concentration of p217+tau present in the sample (step 104), without further concentrating the p217+tau peptides prior to assaying. The blood-based sample may be analyzed using any suitable assay for determining an amount or concentration of p217+tau present in the sample. More particularly, the assay is a highly sensitive and precise assay for measuring p217+tau in plasma. The amount or concentration of p217+tau peptides can be determined using any suitable techniques known in the art, including ELISA and single molecule array platform. According to particular aspects, methods of the present application may use a high sensitivity array platform, such as Quanterix Simoa or MSD S-plex, to measure the amount or concentration of p217+tau peptides in a blood-based sample (specifically a plasma sample). Specifically, the blood-based sample is a plasma sample and the amount or concentration of p217+tau present in the plasma sample is determined using an assay that is able to provide accurate, sensitive and precise quantitative measurements of p217+tau present in plasma samples obtained from cognitively normal subjects (including those who are not amyloid positive), as well as those subjects having or at risk of developing tauopathy or amyloidogenic disease, in particular, those subjects having mild cognitive impairment (MCI) within an acceptable and reliable level of sensitivity. For example, the Lower Limit of Quantification (LLOQ) of the assay may be at or below 0.05 pg/ml, 0.045 pg/ml, 0.04 pg/ml, 0.038 pg/ml or 0.036 pg/ml.

Specifically, the blood-based sample is a plasma sample and the assay used to determine the amount or concentration of p217+tau present in the plasma sample comprises a capture antibody that binds to the p217+tau epitope and a detection antibody, which is labeled with a detectable label (e.g., fluorescent molecule, biotin, etc.) that is directly detectable or detectable via a secondary reaction (e.g., reaction with streptavidin). The capture antibody may be immobilized to a solid phase so that the capture antibody selectively binds to and immobilizes the p217+tau peptides present in the sample to the solid phase. The capture antibody binds to the p217+tau epitope and forms an antibody-peptide complex with the p217+tau peptide. The antibody-peptide complex is subsequently contacted with the detection antibody to bind the detection antibody to the antibody-peptide complex. In one example, the detection antibody is biotinylated. The detectable label on the detection antibody is then detected to determine an amount or concentration of p217+tau peptides in the sample.

In one example, the capture antibody is a monoclonal antibody comprising immunoglobulin heavy chain HCDR1, HCDR2 and HCDR3 having the polypeptide sequences of SEQ ID NOs: 2, 3 and 4, respectively, and immunoglobulin light chain LCDR1, LCDR2 and LCDR3 having the polypeptide sequences of SEQ ID NOs: 5, 6 and 7. In a particular embodiment, the capture antibody is pT3. As used herein, the term “pT3” refers to an antibody that binds to the p217+tau epitope and has a heavy chain variable region amino acid sequence of SEQ ID NO: 8 and a light chain variable region amino acid sequence of SEQ ID NO: 9. In one embodiment, the pT3 monoclonal antibody is expressed by a mouse-hybridoma.

An exemplary detection antibody is a monoclonal antibody that binds to an epitope comprising amino acids 7-20 of human tau protein, wherein the numbering of the positions is according to the numbering in SEQ ID NO: 1. The detection antibody may be a monoclonal antibody comprising immunoglobulin heavy chain HCDR1, HCDR2 and HCDR3 having the polypeptide sequences of SEQ ID NOs: 10, 11, and 12, respectively, and immunoglobulin light chain LCDR1, LCDR2 and LCDR3 having the polypeptide sequences of SEQ ID NOs: 13, 14 and 15. In a particular embodiment, the detection antibody is hT43. As used herein, the term “hT43” refers to a monoclonal antibody that binds to an epitope comprising amino acids 7-20 of human tau protein, wherein the numbering of the positions is according to the numbering in SEQ ID NO: 1, and the antibody has a heavy chain variable region amino acid sequence of SEQ ID NO: 16 and a light chain variable region amino acid sequence of SEQ ID NO: 17.

An exemplary assay for determining the amount of p217+tau present in a plasma sample is described, for example, in WO2022/013286, the entirety of the disclosure of which is incorporated by reference herein.

In step 106, the amount or concentration of p217+tau present in the plasma sample is used to determine whether the subject proceeds with further screening for the clinical trial or alternatively, selected for inclusion in the clinical trial or be indicated as suitable for inclusion in the clinical trial. If the amount or concentration of p217+tau present in the plasma sample of the subject is greater than or equal to a minimum threshold, then the subject is identified as one who is in need of treatment for tauopathy or an amyloidogenic disease. The minimum threshold corresponds to the amount or concentration of p217+tau present in plasma over which subjects present with a “tau positive” pathology, i.e., correspond to those subjects that present with mild cognitive impairment (MCI) and an increased accumulation of tau tangles (e.g., accumulation of singly- or multiply-phosphorylated p217+tau) in the brain, mores specifically, in the bilateral inferior temporal cortex, as compared to a cognitively normal patient. Such an accumulation may correspond to a tau PET image having a SUVR Z-score>1 in the bilateral inferior temporal cortex of the subject. For example, the minimum threshold is 0.075 pg/ml, 0.1 pg/ml, or 0.125 pg/ml. In one example, the minimum threshold is 0.1 pg/ml±0.025 pg/ml. Alternatively, the minimum threshold corresponds to the amount or concentration of p217+tau present in plasma over which subjects tend to correspond to an “amyloid positive” pathology, i.e., corresponding to those subjects that present with mild cognitive impairment (MCI) and an increased accumulation of β-amyloid (e.g., Aβ40 and/or Aβ42) in the bilateral inferior temporal cortex as compared to a cognitively normal patient. The amount or concentration of p217+tau in the plasma sample, as determined in step 104, provides good ability to predict tau positive and/or amyloid positive status of the subject using a minimum threshold of 0.1 pg/ml.

Additionally, if the amount or concentration of p217+tau present in the plasma sample of the subject is less than or equal to a maximum threshold, then the subject is identified as one who is not at late disease progression of tauopathy or amyloidogenic disease so that the treatment is more likely to have an effect and/or any effect by the treatment for tauopathy or an amyloidogenic disease under clinical trial for slowing progression of tauopathy or an amyloidogenic disease can be readily observed. The maximum threshold corresponds to the amount or concentration of p217+tau present in plasma over which subjects tend to have a pathology of widespread accumulation of tau tangles in multiple regions of the brain. For example, such widespread accumulation of tau tangles may correspond to a tau PET imaging having a SUVR Z-score>5 in each of Braak 4, 5, and 6 regions of the brain of the subject. In one example, the maximum threshold is 0.225 pg/ml, 0.25 pg/ml, 0.275 pg/ml, 0.3 pg/ml. In another example, the maximum threshold is 0.25 pg/ml±0.025 pg/ml.

In one embodiment, if the amount or concentration of p217+tau present in the plasma sample is ≥ the minimum threshold and ≤ the maximum threshold, then the subject proceeds with further screening for the clinical trial, or alternatively, selected for inclusion in the clinical trial or be indicated as suitable for inclusion in the clinical trial (step 108). If the amount or concentration of p217+tau present in the plasma sample is < the minimum threshold or > the maximum threshold, then the subject is excluded from the clinical trial (step 110). For example, if the amount or concentration of p217+tau present in the plasma sample is ≥0.1±0.025 pg/ml and ≤0.25±0.025 pg/ml, then the subject proceeds with further screening for the clinical trial or alternatively, selected for inclusion in the clinical trial or be indicated as suitable for inclusion in the clinical trial (step 108). If the amount or concentration of p217+tau present in the plasma sample is <0.1±0.025 pg/ml or >0.25±0.025 pg/ml, then the subject is excluded from the clinical trial (step 110). In another example, if the concentration of p217+tau present in the plasma sample is from 0.075 pg/ml to 0.275 pg/ml, from 0.075 pg/ml to 0.25 pg/ml, from 0.075 pg/ml to 0.225 pg/ml, from 0.1 pg/ml to 0.275 pg/ml, from 0.1 pg/ml to 0.25 pg/ml, from 0.1 pg/ml to 0.225 pg/ml, from 0.125 pg/ml to 0.275 pg/ml, from 0.125 pg/ml to 0.25 pg/ml, or from 0.125 pg/ml to 0.225 pg/ml, then the subject proceeds with further screening for the clinical trial or alternatively, selected for inclusion in the clinical trial or be indicated as suitable for inclusion in the clinical trial (step 108). If the amount or concentration of p217+tau present in the plasma sample is outside of the ranges identified above, then the subject is excluded from the clinical trial (step 110).

In another aspect of the present application, a method 200 for pre-screening or screening a human subject for a clinical trial for treatment of tauopathy or an amyloidogenic disease is provided. The method 200 comprises a first step 202 of obtaining a blood-based sample, and more specifically, a plasma sample, from the human subject in the same manner as step 102, and step 204, which is similar to step 104 for determining an amount or concentration of p217+tau present in the sample. However, in step 204, each sample obtained in step 202 may be assayed once or may be divided into aliquots and assayed a plurality of times (e.g., twice, thrice, etc.) to generate a plurality of measurements of amount or concentration of p217+tau detected from the same sample.

In step 206, if the sample is assayed a plurality of times, then the resulting multiple measurements of amount or concentration of p217+tau detected from the same sample are analyzed to determine whether the sample is assayed with sufficient precision. For example, a percent coefficient of variation (% CV) may be determined for the multiple measurements obtained from the same sample. If the sample is assayed only once, % CV is not determined for the sample. If the % CV is less than a threshold CV, then the method 200 proceeds to step 208. If the % CV determined for the multiple measurements is greater than the threshold CV or if the sample is assayed only once, then the method 200 proceeds to step 210. The threshold CV may be 25% CV, 20% CV or 15% CV. In one particular example, if the % CV is <20%, then the method 200 proceeds to step 208. If the % CV is ≥20% or if the sample is assayed only once, then the method 200 proceeds to step 210.

In step 208, if the mean amount or concentration of p217+tau across multiple measurements of the sample is ≥ the minimum threshold and ≤ the maximum threshold, then the subject proceeds with further screening for the clinical trial, or alternatively, selected for inclusion in the clinical trial or be indicated as suitable for inclusion in the clinical trial. If the mean amount or concentration of p217+tau across multiple measurement of the sample is < the minimum threshold or > the maximum threshold, then the subject is excluded from the clinical trial. The minimum threshold and the maximum threshold are defined in the same manner as described above for step 106. In one example, if the mean amount or concentration of p217+tau across multiple measurements of a plasma sample is ≥0.1±0.025 pg/ml and ≤0.25±0.025 pg/ml, then the subject proceeds with further screening for the clinical trial or alternatively, selected for inclusion in the clinical trial or be indicated as suitable for inclusion in the clinical trial. If the mean amount or concentration of p217+tau across multiple measurements of the plasma sample is <0.1±0.025 pg/ml or >0.25±0.025 pg/ml, then the subject is excluded from the clinical trial. In another example, if the mean amount or concentration of p217+tau across multiple measurements of the plasma sample is from 0.075 pg/ml to 0.275 pg/ml, from 0.075 pg/ml to 0.25 pg/ml, from 0.075 pg/ml to 0.225 pg/ml, from 0.1 pg/ml to 0.275 pg/ml, from 0.1 pg/ml to 0.25 pg/ml, from 0.1 pg/ml to 0.225 pg/ml, from 0.125 pg/ml to 0.275 pg/ml, from 0.125 pg/ml to 0.25 pg/ml, or from 0.125 pg/ml to 0.225 pg/ml, then the subject proceeds with further screening for the clinical trial or alternatively, be considered as suitable for inclusion in the clinical trial (step 210). If the mean amount or concentration of p217+tau across multiple measurements of the plasma sample is outside of the ranges identified above, then the subject is excluded from the clinical trial (step 212).

In step 220, the mean amount or concentration of p217+tau across multiple measurements of the sample or the single measurement of amount or concentration of p217+tau in a plasma sample is used to determine which of three different steps the method 200 will proceed next. First, if the mean amount or concentration of p217+tau across multiple measurements of the plasma sample or the single measurement of amount or concentration of p217+tau in the plasma sample is within a range that is ±Δ of the minimum threshold or ±Δ of the maximum threshold, then the method 200 proceeds to step 222. For example, Δ is 0.01 pg/ml, 0.015 pg/ml, 0.02 pg/ml, 0.025 pg/ml, 0.03 pg/ml, or 0.035 pg/ml.

In step 222, the sample is retested in the same manner as described in step 204 to obtain a further measurement of an amount or concentration of p217+tau present in the sample. In step 224, if the retest data provides similar quantitative results as prior measurements (e.g., within ± standard deviation to mean value) and improves (i.e., decreases % CV) for the sample, then the mean amount or concentration of p217+tau across all measurements (including the retest) is further analyzed according to the criteria of step 208 to determine whether to proceed to step 210 or exclude the subject from the clinical trial (step 212).

Next, if the mean amount or concentration of p217+tau across multiple measurements of the plasma sample or the single measurement of amount or concentration of p217+tau in the plasma sample is <LLOQ of the assay, then the subject is excluded from the clinical trial (step 212). Retest is not needed in this scenario as the amount or concentration of p217+tau in the plasma sample is below the detectable range of the assay and thus unlikely to yield a superior CV %.

Lastly, if the mean amount or concentration of p217+tau across multiple measurements of the plasma sample or the single measurement of amount or concentration of p217+tau in the plasma sample is:
(1) ≥LLOQ and <minimum threshold −Δ,
(2) >minimum threshold +Δ and <maximum threshold −Δ, or
(3) >maximum threshold +Δ,
   wherein Δ is as identified above, then the method 200 proceeds to step 208. If the amount or concentration of p217+tau in the plasma sample falls within any of the 3 criteria identified above, the method 200 may optionally include a retesting step (not shown) to improve precision of the mean amount or concentration of p217+tau across multiple measurements and/or reduce % CV for the measurements obtained for the sample.

In a particular embodiment, the minimum threshold is 0.1 pg/ml, the maximum threshold is 0.25 pg/ml, Δ is 0.025 pg/ml, and LLOQ of the assay is 0.036 pg/ml. In step 208 of this embodiment, if the mean concentration of p217+tau across multiple measurements of the plasma sample is ≥0.1 pg/ml and ≤0.25 pg/ml, then the subject proceeds with further screening for the clinical trial, or alternatively, be considered as suitable for inclusion in the clinical trial (step 210). In contrast, if the mean concentration of p217+tau across multiple measurements of the plasma sample is <0.1 pg/ml or >0.25 pg/ml, then the subject is excluded from the clinical trial (step 212).

In step 220 of this embodiment, the mean concentration of p217+tau across multiple measurements of the plasma sample or the single measurement of concentration of p217+tau in the plasma sample is used to determine which of three different steps the method 200 will proceed next. Specifically, if the mean concentration of p217+tau across multiple measurements of the plasma sample or the single measurement of concentration of p217+tau in the plasma sample is within 0.075 to 0.125 pg/ml or 0.225 to 0.275 pg/ml, then the method 200 proceeds to step 222. If the mean concentration of p217+tau across multiple measurements of the plasma sample or the single measurement of concentration of p217+tau in the plasma sample is <0.036 pg/ml, then the subject is excluded from the clinical trial (step 212). If the mean concentration of p217+tau across multiple measurements of the plasma sample or the single measurement of concentration of p217+tau in the plasma sample is:
(1) ≥0.036 pg/ml and <0.075 pg/ml,
(2) >0.125 pg/ml and <0.225 pg/ml, or
(3) ≥0.275 pg/ml,
   then the method 200 proceeds to step 208.

In some embodiments, subjects identified in step 108 or 210 may be directed to additional clinical tests, such as for example, PET imaging and/or CSF collection, to further assess brain pathologies of these subjects. The additional clinical tests may be used to confirm that the subject is need of a treatment for tauopathy or an amyloidogenic disease (e.g., having mild cognitive impairment (MCI) and an increased accumulation of tau tangles in the brain as compared to a cognitively normal patient), and that the subject is not at late disease progression of tauopathy or amyloidogenic disease (e.g., presenting with pathology of widespread accumulation of tau tangles or amyloid fibrils in multiple regions of the brain).

For example, the subjects identified in step 108 or 210 may be directed to tau PET imaging using a tau-specific tracer (e.g., a p217+tau specific tracers) or amyloid PET using an amyloid-specific radiotracers (e.g., Aβ40 or Aβ42 specific radiotracers). The PET data obtained may be analyzed by a computing device to confirm that the subject is tau positive or amyloid positive. For example, the computing device may analyze tau PET data to confirm that the subject has an increased accumulation of tau tangles (e.g., accumulation of singly-or multiply-phosphorylated p217+tau) in the brain, mores specifically, in the bilateral inferior temporal cortex, as compared to a cognitively normal patient. More specifically, the tau PET data may be analyzed to confirm that the subject has a SURVR Z-score>1 in the bilateral inferior temporal cortex. In another example, amyloid PET data may be analyzed to confirm that the subject has an increased accumulation of amyloid fibrils in the brain, mores specifically, in the bilateral inferior temporal cortex, as compared to a cognitively normal patient.

The computing device may also analyze the PET data obtained to confirm that the subject is not at late disease progression of tauopathy or amyloidogenic disease (e.g., presenting with pathology of widespread accumulation of tau tangles or amyloid fibrils in multiple regions of the brain). For example, tau PET data may be analyzed to confirm that the subject does not have widespread accumulation of tau tangles in the brain. More specifically, the tau PET data may be analyzed to confirm that the subject does not have a SUVR Z-score>5 in each of Braak 4, 5 and 6 regions of the brain of the subject. In another example, amyloid PET data may be analyzed to confirm that the subject does not have widespread accumulation of amyloid fibrils in multiple regions of the brain, such as, Braak 4, 5 and 6 regions of the brain of the subject.

In one embodiment, a method of screening a human subject for a clinical trial for treatment of tauopathy or an amyloidogenic disease is provided. The method comprises obtaining a plasma sample from the subject and determining an amount or concentration of p217+tau present in the sample in the same manner as steps 102 and 104 described above. When the amount or concentration of p217+tau present in the plasma sample is greater than or equal to a minimum threshold and less than or equal to a maximum threshold, the method proceeds to obtaining PET data of the brain of the subject generated with a tau-specific or amyloid specific radioactive tracer. The minimum threshold and the maximum threshold are defined in the same manner as described above for step 106. The PET data is then analyzed to determine whether the subject is tau positive or amyloid positive, and whether the subject has widespread tau tangles or widespread amyloid fibrils. The subject is indicated as suitable for inclusion in the clinical trial when:
(i) the concentration of p217+tau present in the plasma sample is greater than or equal to the minimum threshold and less than or equal to the maximum threshold,
(ii) the PET data indicates the subject is tau positive or amyloid positive, and
(iii) the PET data indicates the subject does not have without having widespread tau tangles or widespread amyloid fibrils.

Furthermore, those subjects indicated as suitable for inclusion in the clinical trial may be further stratified into High and Low groups based on SUVR in the Braak 4 region generated from the PET data. For example, subjects are stratified into the High group when SUVR in the Braak 4 region is >4.62 and into the Low group when SUVR in the Braak 4 region is ≤4.62.

Those skilled in the art will understand that the exemplary computer-implemented embodiments described herein may be implemented in any number of manners, including as a separate software module, as a combination of hardware and software, etc. For example, the exemplary methods may be embodiment in one or more programs stored in a non-transitory storage medium and containing lines of code that, when compiled, may be executed by one or more processor cores or a separate processor. A system according to one embodiment comprises a plurality of processor cores and a set of instructions executing on the plurality of processor cores to perform the exemplary methods discussed above. The processor cores or separate processor may be incorporated in or may communicate with any suitable electronic device, for example, on board processing arrangements within the device or processing arrangements external to the device, e.g., a mobile computing device, a smart phone, a computing tablet, a computing device, etc., that may be in communications with at least a portion of the device.

### EXAMPLES

The following examples are to further illustrate the nature of the invention. It should be understood that the following examples do not limit the invention and that the scope of the invention is to be determined by the appended claims.

### Example 1:

Example 1 reports data on performance of a plasma 217+tau assay as a prescreening tool for identifying subjects who are likely tau positive for inclusion in a clinical trial for treatment of tauopathy. The assay is performed using Quanterix's high sensitivity Simoa array platform with the pT3 antibody as the capture antibody and the hT43 antibody attached to a detectable label as the detection antibody. In addition, Example 1 describes how the assay can be used to enrich for low tau PET stratum patients for inclusion in a clinical trial for treating or slowing progression of tauopathy.

In Example 1, the plasma p217+tau assay was run on screened samples from subjects presenting with MCI in weekly batches. Technical performance across 58 batches was evaluated. Subjects presenting with plasma p217+tau levels above a pre-specified cutoff of 0.1 pg/ml progressed to Tau PET (using MK-6240 as tracer) screening. Concentrations of plasma p217+tau in this population and prevalence of tau positive status in subjects having a concentration of p217+tau in plasma ≥0.1 pg/ml was studied in Example 1.

A total of 55 batches of plasma samples were screened using the p217+tau assay of Example 1 described above. A panel of 3 peptide QCs (0.1, 0.4, and 1.6 pg/ml) were run in duplicate in each batch revealing excellent intra-run precision (average CV=6.0, 4.5, and 5.5%, respectively) and inter-run precision (7.6, 6.6, and 13.3% CV, respectively). Precision was also acceptable with endogenous samples, as amongst N=725 plasma samples the mean CV was 7.9% (0-120% range), with an estimated LLOQ of 0.030 pg/ml (based on mean concentration where CV>20%).

Of the 787 MCI patients in which plasma p217+tau was measured, 72% had levels ≥0.1 pg/ml. Those patients who had plasma p217+tau concentrations ≥0.1 pg/ml were then slated to have tau PET imaging performed. Of the 346 patients imaged, 86% were tau positive and 64% satisfied the criteria for trial tau PET eligibility criteria of having a SUVR Z-score>1 in the bilateral inferior temporal cortex of the subject while not having a SUVR Z-score>5 in each of Braak 4, 5, and 6 regions of the brain of the subject.

Tau PET data was also used to stratify patients into High and Low groups based on SUVR in the Braak 4 region. While assay screening performed as expected, 59% of eligible patients were in the high stratum. ROC analysis shows the plasma p217+tau assay can predict patient stratum with an AUC of 0.8. Therefore, to correct the stratification imbalance, after 1 year of data collection, an upper threshold of plasma p217+tau concentration (i.e., ≤0.25 pg/ml) was implemented to enrich for patients having low to moderate progression of tauopathy.

The plasma p217+tau assay of Example 1 showed good precision within and between batches and demonstrated the ability to enrich populations for both tau positivity and tau stratification. The assay can serve as a tool to enable faster and more efficient enrollment for a clinical trial for a treatment for Alzheimer's disease or to identify incipient Alzheimer's disease in the general population.

### Example 2:

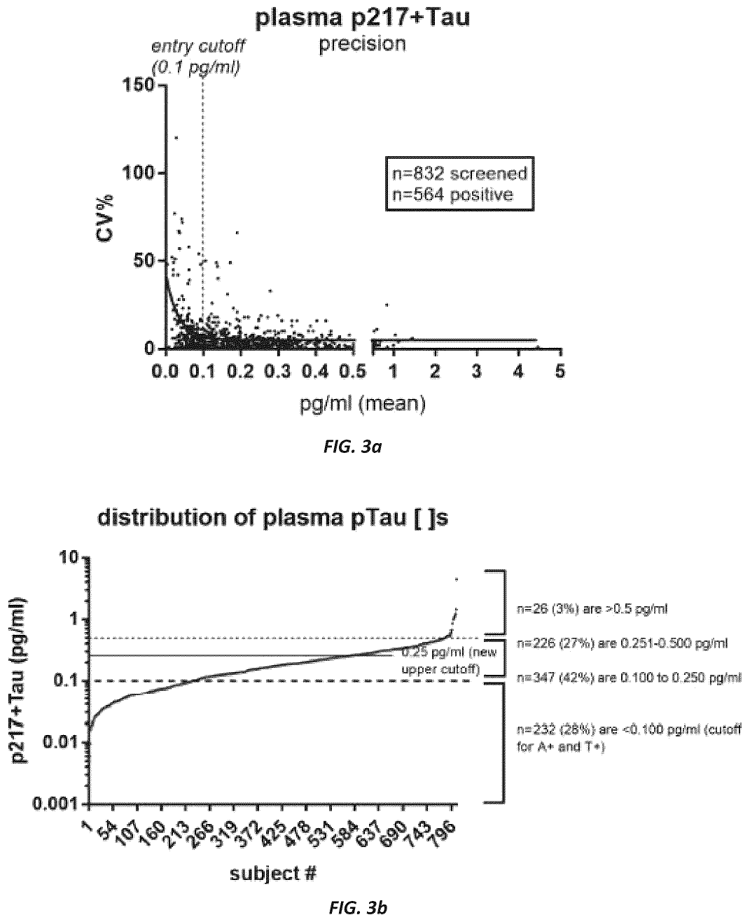

Example 2 provide additional data on the performance of the plasma p217+tau assay for patients screened in the same clinical trial as Example 1. The data of Example 2 includes more samples than the data reported in Example 1. FIG. 3a shows precision (% CV) of the plasma p217+tau assay across different concentrations of p217+tau detected from plasma samples collected from 832 patients screened for the clinical trial. FIG. 3b shows a distribution of concentrations of p217+tau detected by the p217+tau assay from plasma samples collected from 831 patients screened for the clinical trial.

### Example 3: Correlations Between Simoa Plasma p217+Tau and Tau PET in Participants Screened for a Clinical Trial

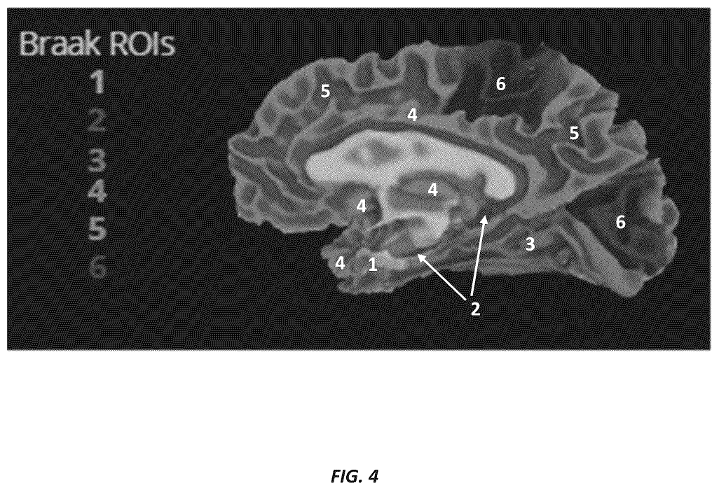

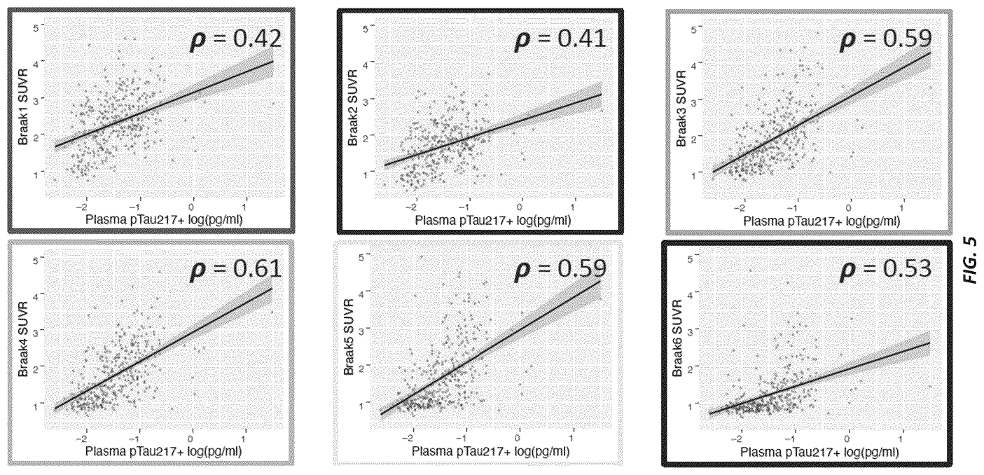

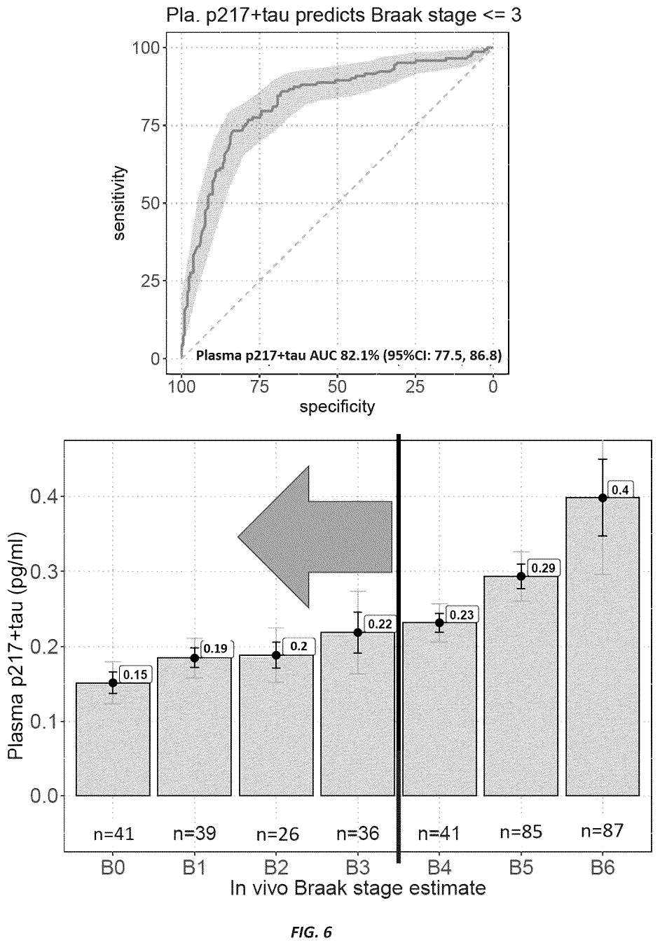

Example 3 provides a report on correlations observed between plasma p217+tau and 18F-MK-6240 tau PET uptake across brain regions. Simoa-based plasma p217+tau assay was conducted in weekly batches (Triana-Baltzer et al., Alzheimers Dement (Amst). 2021). Participants with levels exceeding 0.1 pg/ml were then scanned to confirm the presence of NFT on tau PET. NFT levels were quantified using 18F-MK-6240 SUVR in Braak Regions of Interest (ROI) (Scholl et al. Neuron 2016) (reference region: cerebellar gray). Braak ROIs are shown in FIG. 4. FIG. 5 shows significant (p<10⁻¹⁵) correlations between plasma p217+tau and MK6240 across all six (6) Braak ROIs. FIG. 6 shows that plasma p217+tau can predict in-vivo Braak stage of 3 or lower with AUC of 82%. Non-parametric Spearman correlations between plasma p217+tau levels and average SUVR over each of the 6 ROIs used for Braak staging were obtained. The results reported herein in Example 3 are from the first set of 355 participants for which the Tau PET analysis was completed.

As shown herein, significant correlations were found between the plasma p217+tau and tau PET in a clinical trial setting. The correlations were consistent with an independent observational study in cognitively impaired individuals (Dore et al. Alzheimer's Dement. 2022). The results demonstrate consistent performance and utility of plasma p217+tau as a pre-screen in Alzheimer's Disease (AD) trials. Simoa plasma p217+tau levels are consistently correlated with tau PET and can be used as a pre-screen in clinical trials to reduce participant burden and improve screening efficiency.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

What is claimed is:

1. A method of pre-screening a human subject for a clinical trial for treatment of tauopathy or an amyloidogenic disease, comprising:
obtaining a plasma sample from the subject;
determining a concentration of p217+tau present in the plasma sample; and
indicating the subject for further screening for the clinical trial when the concentration of p217+tau present in the plasma sample is greater than or equal to a minimum threshold and less than or equal to a maximum threshold,
wherein the minimum threshold corresponds to an amount of p217+tau in plasma over which subjects present with mild cognitive impairment (MCI) and an increased accumulation of tau tangles in the brain as compared to a cognitively normal patient, and
wherein the maximum threshold corresponds to an amount of p217+tau present in plasma over which subjects present with pathology of widespread accumulation of tau tangles in multiple regions of the brain.

2. The method of claim 1, wherein the minimum threshold is from about 0.075 pg/ml to about 0.125 pg/ml.

3. The method of claim 1, wherein the maximum threshold is from about 0.225 pg/ml to about 0.275 pg/ml.

4. The method of claim 1, wherein the indicating step comprises indicating the subject for further screening for the clinical trial when the concentration of p217+tau present in the plasma sample is ≥0.1 pg/ml and ≤0.25 pg/ml.

5. The method of claim 1, wherein the determining step comprises:
contacting the plasma sample with a capture antibody directed against a p217+tau epitope to bind the capture antibody to p217+tau peptides in the plasma sample to form antibody-peptide complexes;
contacting the antibody-peptide complexes with a detection antibody to bind the detection antibody to the antibody-peptide complexes; and
detecting the detection antibody to determine an amount of the p217+tau peptides in the plasma sample.

6. The method of claim 2, wherein the capture antibody binds to an epitope containing amino acids 210-220 of human tau protein.

7. The method of claim 2, wherein the detection antibody binds to an epitope comprising amino acids 7-20 or 116-127 of human tau protein.

8. The method of claim 3, wherein the capture antibody is pT3.

9. The method of claim 4, wherein the detection antibody is hT43.

10. The method of claim 1, wherein the concentration of p217+tau is determined using an assay having a LLOQ<0.04 pg/ml.

11. The method of claim 1, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

12. The method of claim 11, wherein the tauopathy is Alzheimer's disease.

13. A method of screening a human subject for a clinical trial for treatment of tauopathy or an amyloidogenic disease, comprising:
obtaining a plasma sample from the subject;
determining a concentration of p217+tau present in the plasma sample;
obtaining PET data of the brain of the subject generated with a tau-specific or amyloid-specific radioactive tracer when the concentration of p217+tau present in the plasma sample is greater than or equal to a minimum threshold and less than or equal to a maximum threshold;
analyzing the PET data to determine whether the subject is tau positive or amyloid positive, and whether the subject has widespread tau tangles or widespread amyloid fibrils; and
indicating the subject as suitable for inclusion in the clinical trial when:
(iv) the concentration of p217+tau present in the plasma sample is greater than or equal to the minimum threshold and less than or equal to the maximum threshold,
(v) the PET data indicates the subject is tau positive or amyloid positive, and
(vi) the PET data indicates the subject does not have without having widespread tau tangles or widespread amyloid fibrils,
wherein the minimum threshold corresponds to an amount of p217+tau in plasma over which subjects present with mild cognitive impairment (MCI) and an increased accumulation of tau tangles in the brain as compared to a cognitively normal patient, and
wherein the maximum threshold corresponds to an amount of p217+tau present in plasma over which subjects present with pathology of widespread accumulation of tau tangles in multiple regions of the brain.

14. The method of claim 13, wherein the minimum threshold is from about 0.075 pg/ml to about 0.125 pg/ml.

15. The method of claim 13, wherein the maximum threshold is from about 0.225 pg/ml to about 0.275 pg/ml.

16. The method of claim 13, wherein the indicating step comprises indicating the subject for further screening for the clinical trial when the concentration of p217+tau present in the plasma sample is ≥0.1 pg/ml and ≤0.25 pg/ml.

17. The method of claim 13, wherein the determining step comprises:
contacting the plasma sample with a capture antibody directed against a p217+tau epitope to bind the capture antibody to p217+tau peptides in the plasma sample to form antibody-peptide complexes;
contacting the antibody-peptide complexes with a detection antibody to bind the detection antibody to the antibody-peptide complexes; and
detecting the detection antibody to determine an amount of the p217+tau peptides in the plasma sample.

18. The method of claim 17, wherein the capture antibody binds to an epitope containing amino acids 210-220 of human tau protein.

19. The method of claim 17, wherein the detection antibody binds to an epitope comprising amino acids 7-20 or 116-127 of human tau protein.

20. The method of claim 18, wherein the capture antibody is pT3.

21. The method of claim 19, wherein the detection antibody is hT43.

22. The method of claim 13, wherein the concentration of p217+tau is determined using an assay having a LLOQ<0.04 pg/ml.

23. The method of claim 13, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

24. The method of claim 23, wherein the tauopathy is Alzheimer's disease.
